(19) United States
(12) Patent Application Publication
(10) Pub. No.: US 2022/0288034 A1
(43) Pub. Date: Sep. 15, 2022

(54) METHOD OF REDUCING ABERRANT IMMUNE RESPONSE OR RISK OF PROGRESSION IN COVID-19

(71) Applicant: CELLTAXIS, LLC, Atlanta, GA (US)

(72) Inventors: Eric B. Springman, Atlanta, GA (US); Ralph Grosswald, Alpharetta, GA (US); Sanjeev Ahuja, Carmel, IN (US)

(21) Appl. No.: 17/687,816

(22) Filed: Mar. 7, 2022

Related U.S. Application Data
(60) Provisional application No. 63/158,501, filed on Mar. 9, 2021.

(51) Int. Cl. A61K 31/422; A61P 37/02; A61K 45/06; A61K 9/00

(52) U.S. Cl. CPC A61K 31/422; A61P 37/02; A61K 45/06; A61K 9/0053

(57) ABSTRACT

The invention is directed to methods of treating a patient that is or has been infected with COVID-19 comprising the use of acebilustat. The methods include reducing the risk of COVID-19 progression, reducing or preventing aberrant immune response, reducing or preventing COVID-19 mediated lung injury, and/or treating late sequelae of COVID-19.

### RELATED APPLICATION

This application claims the benefit of U.S. Provisional Application No. 63/158,501 filed Mar. 9, 2021. The entire contents of the above-referenced application are incorporated by reference herein.

### BACKGROUND OF THE INVENTION

Symptoms of coronavirus disease 2019 (COVID-19; caused by SARS-CoV-2 infection) include fever, dry cough and muscle aches (Wu et al. (2020), Risk factors associated with acute respiratory distress syndrome and death in patients with coronavirus disease 2019 pneumonia in Wuhan China, JAMA Internal Medicine 180(7):934-943; Merad et al. (2020), Pathological inflammation in patients with COVID-19: a key role for monocytes and macrophages, Nature Reviews Immunology 20: 355-362). While some patients experience mild disease, others develop severe illness which, in some cases, progresses to acute respiratory distress syndrome or organ failure (Id.). Based on a 2019 study from China, about 80% of COVID-19 patients had mild to moderate disease (mild symptoms up to mild pneumonia) while about 14% patients developed severe disease (symptoms including dyspnea, hypoxia, and/or more than 50% lung involvement on imaging) (www.cdc.gov/coronavirus/2019-ncov/hcp/clinical-guidance-management-patients.html). About 5% of patients developed critical disease characterized by respiratory failure, shock, and/or multiorgan system dysfunction (Id.).

There is evidence that aberrant immune response (also referred to in the literature as immune dysregulation and/or hyperinflammation) is a key feature of disease progression (see, for example, Lucas et al. (2020), Longitudinal analyses reveal immunological misfiring in severe COVID-19, Nature 584: 463-469 and Hussman (2020), Cellular and Molecular Pathways of COVID-19 and Potential Points of Therapeutic Intervention, Frontiers in Pharmacology, https://doi.org/10.3389/fphar.2020.01169). Specifically, excessive immune infiltration without or in advance of substantial antibody response to the virus appears to confer high risk for progression. This is corroborated by the finding that patients with COPD are among those at highest risk of disease progression (Sin et al. (2020), COVID-19 in COPD: A Growing Concern, EClinicalMedicine 100546). For their part, neutrophils and, to a large extent, macrophages are not major contributors to the development of an effective antibody response to viral infection. However, they are significant contributors to the acute responses to injury/damage in the lung, leading to inflammation. For instance, in acute lung injury (ALI), macrophages and neutrophils are thought to serve a deleterious role in progression to ARDS. Similarly, in COVID-19, neutrophil and macrophage infiltrates have been associated with worse outcomes (Borges et al. (2020), COVID-19 and Neutrophils: The Relationship between Hyperinflammation and Neutrophil Extracellular Traps, Mediators of Inflammation https://doi.org/10.1155/2020/8829674; Merad et al. (2020), Pathological inflammation in patients with COVID-19: a key role for monocytes and macrophages, Nature Reviews Immunology 20: 355-362; Cavalcante-Silva et al. (2021), Neutrophils and COVID-19: The road so far, Int Immunopharmacol. 10.1016/j.intimp.2020.107233). The presentation of these inflammatory infiltrates and edema prior to progression to ARDS in COVID-19 patients is reminiscent of key features of the exudative phase of ALI.

There remains a need in the art for therapeutic treatments that reduce the risk of acute lung injury or ARDS and/or reduce the risk of disease progression in COVID-19 patients.

### SUMMARY OF THE INVENTION

The present invention is directed to methods of treating a patient that is or has been infected with COVID-19. The methods encompass reducing the risk of COVID-19 progression; reducing or preventing aberrant immune response, or decreasing the risk of aberrant immune response in a COVID-19 patient; reducing or preventing COVID-19 mediated lung injury, or decreasing the risk of COVID-19-mediated lung injury; treating moderate to severe COVID-19; or treating late sequelae of COVID-19 (also referred to in the literature as “long haul” COVID-19 or post-COVID-19 syndrome).

The methods comprise administering to the patient an effective amount of acebilustat. For example, the acebilustat can be administered at least once a day, for example orally. In certain embodiments, the methods comprise orally administering to patients acebilustat at a total daily dose of about 200 mg or less, about 150 mg or less, about 100 mg or less, about 50 mg or less, from about 50 mg to about 100 mg, about 100 mg, or about 50 mg. In certain embodiments, the patient has from mild or moderate disease and/or the patient is at risk for disease progression or has one or more risk factors for disease progression.

### DETAILED DESCRIPTION OF THE INVENTION

A description of preferred embodiments of the invention follows.

As used herein, the words “a” and “an” are meant to include one or more unless otherwise specified. For example, the term “an additional therapeutic agent” encompasses both a single additional therapeutic agent and a combination of two or more additional therapeutic agents.

It is to be understood that when the range of the dose or amount of a drug or active ingredient (e.g., acebilustat) is described as “between” a low end of the range and “between” a high end of the range, the range is meant to include both, the low end and the high end as well as doses in between the low and high ends. For example, for “a dose between about 50 mg and about 100 mg,” it is to be understood that the range includes the low end of the range, about 50 mg, and the high end of the range, about 100 mg, as well as the doses in between, for example, about 75 mg. In addition, “a dose of about 50 mg or less” is intended to include the about 50 mg dose as well as doses less than about 50 mg.

The term “about” as used herein, in reference to a numerical value or range, allows for a degree of variability in the value or range, for example, within 10%, within 5%, or within 4%, or within 2% of the value or range.

As used herein, “COVID-19” is a disease or syndrome caused by SARS-CoV-2 infection. Thus, a patient that is or has been infected with COVID-19 is a patient that is or has been infected with the SARS-CoV-2 virus. A patient suffering from COVID-19 is a patient that is or has been infected with COVID-19 or is suffering from the consequences or late sequelae of COVID-19.

The present invention is at least partially based on the appreciation that inhibition of LTB4 by acebilustat has therapeutic potential in treating COVID-19 patients, and specifically in reducing the risk of progression or aberrant immune response (including, for example reducing the risk of ARDS or ALI or COVID-19 induced lung injury). The methods can, for example, be used to reduce the risk of progression and/or reduce aberrant immune response and/or reduce lung injury patients with mild or moderate disease. The methods can also be used to treat patients with moderate to severe COVID-19 and include reducing lung injury and/or reducing aberrant immune response. The methods can also be used to treat a patient suffering from late sequelae of COVID-19.

Acebilustat and other LTA4-h inhibitors have been described, for example, in U.S. Pat. No. 7,737,145, U.S. Pat. No. 9,820,974, and U.S. Patent Application Publication No. 20100210630A1, the contents of each of which are incorporated by reference herein. The chemical name of acebilustat is 4-{[(1 S,4S)-5-({4-[4-oxazol-2-yl-phenoxy]phenyl}methyl)-2,5-diazabicyclo[2.2.1]heptan-2-yl]methyl}benzoic acid (also referred to as CTX-4430). Acebilustat is a potent inhibitor of Leukotriene A4 Hydrolase (LTA4H), the rate-limiting enzyme in production of leukotriene B4 (LTB4).

LTB4 has long been associated with severity and outcomes in ALI, ARDS and multisystem organ failure (MSOF) (Auner et al. (2012), Mediators of Inflammation 2012;2012:536156. doi: 10.1155/2012/536156. Epub 2012 Mar 7. PMID: 22529525; PMCID: PMC3317019). This association is recapitulated in many models of lung injury, including virus-induced acute lung injury, ventilation injury, endotoxin induced lung injury, aspiration pneumonia, hemorrhagic shock, tobacco smoking, and injury induced by various chemical insults (see, for example, Stormann et al. (2017), Prostaglandins, Leukotrienes and Essential Fatty Acids 127: 25-31; Monteiro et al. (2014), American Journal of Respiratory Cell and Molecular Biology 50(1): 87-95). In these models, it was shown that LTB4 presents as an early mediator and marker of disease progression and further, that inhibitors of the LTB4 pathway are effective in these models.

Reports to date suggest that about 20-30% of patients hospitalized for COVID-19 will require intensive care for respiratory support. These are largely represented by the estimated 17-29% of hospitalized patients who develop acute respiratory distress syndrome (ARDS). Overall, 40-70% of hospitalized patients are estimated to require invasive ventilation support. As discussed above, there is evidence that aberrant immune response is a key feature of disease progression.

The present invention is directed to the treatment of COVID-19 patients with acebilustat. In certain aspects, the patients are those with mild or moderate disease and/or that are a risk of progression (e.g., has one or more risk factors). In yet additional aspects, the patient has mild disease and/or is at risk for progression. Specifically, the present invention encompasses methods of reducing the risk of COVID-19 progression; methods of reducing, preventing, or decreasing the risk of aberrant immune response in a COVID-19 patient; and methods of reducing, preventing, or decreasing the risk of COVID-19-mediated lung injury comprising administering to the patient an effective amount of acebilustat. The methods also include treatment of patients with moderate to severe COVID-19, including reducing aberrant immune response and/or reducing COVID-19 mediated lung injury. The methods can also include treatment of patients that have late sequelae of COVID-19. The methods of the invention comprise administration of an effective oral dose of acebilustat (also known as CTX-4430) to human patients. This compound and methods for the preparation thereof have been described in detail in U.S. Pat. No. 7,737,145, U.S. Pat. No. 9,820,974, and U.S. Patent Application Publication No. 20100210630A1, the contents of each of which are incorporated by reference herein. Acebilustat has the chemical structure shown below:

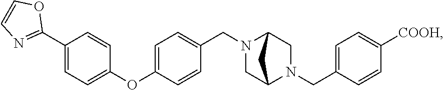 [SMILES: O=C(O)c1ccc(CN2C[C@@H]3C[C@H]2CN3Cc2ccc(Oc3ccc(-c4ncco4)cc3)cc2)cc1]

In vitro, acebilustat inhibits the epoxide hydrolase enzymatic activity of LTA4H with an IC50 of 6.3 ng/mL for LTB4 production. In human whole blood tested ex vivo, acebilustat inhibits LTB4 production with an approximate IC50 of 30.8 ng/mL. Acebilustat 48 ng/mL has also been shown to reduce neutrophil swarming in vitro by 80% in response to factors present in human cystic fibrosis (CF) sputum. In pharmacodynamic studies in humans, acebilustat inhibits LTB4 production with an estimated in vivo EC50 of 93 ng/mL. In CF patients, sputum white blood cells were decreased by 31% from Baseline in all treated subjects (doses of 50 or 100 mg) and by 60% from Baseline in the 100 mg acebilustat group. Sputum neutrophils decreased by 34% in all treated patients (doses of 50 to 100 mg) and by 65% in the 100 mg group. In a recent Phase II study of adult patients with CF, acebilustat showed promise in reducing the rate of pulmonary exacerbations over the course of 48 weeks of treatment with no evidence of increased risk of infection (described, for example, in U.S. Pat. No. 10,898,484; the contents of which are expressly incorporated by reference herein). This effect was most notable in patients with early disease.

The methods described herein comprise administration of an effective amount of acebilustat to a COVID-19 patient. As discussed above, a COVID-19 patient is or has been infected with the SARS-CoV-2 virus. The COVID-19 patient can be asymptomatic. The COVID-19 patient can, in some embodiments, be a symptomatic patient (a patient that reports or experiences one or more symptoms of COVID-19). In additional aspects the COVID-19 patient is one that has been diagnosed as positive based on a laboratory test. Current laboratory tests detect SARS-CoV-2 RNA or antigen in respiratory specimen, e.g., nasopharynx samples (cdc.gov/coronavirus/2019-ncov/hcp/clinical-guidance-management-patients.html). It has been reported that detection of SARS-CoV-2 RNA in blood may be a marker of severe disease.

The present invention encompasses administration of acebilustat to patients that are suffering from or that have mild to moderate COVID-19. In certain aspects, the patient is suffering from or has mild COVID-19.

The invention also encompasses administration of acebilustat to a patient that is suffering from or has moderate to severe COVID-19.

In some examples, an effective amount of acebilustat administered orally can be 200 mg or less. The invention encompasses oral administration of about 200 mg or less acebilustat to the COVID-19 patient. In certain aspects, the patient is administered 200 mg of acebilustat; for example, chronic oral administration (e.g., for more than about one day, for at least about one week, for at least about two weeks, for at least about three weeks, for at least about one month, and/or throughout the patient's treatment). The invention encompasses oral administration of about 100 mg acebilustat to said patient; for example, chronic oral administration (e.g., for more than about one day, for at least about one week, for at least about two weeks, for at least about three weeks, for at least about one month, and/or throughout the patient's treatment). The invention also encompasses administration of 50 mg acebilustat to said patient; for example, chronic oral administration (e.g., for more than about one day, for at least about one week, for at least about two weeks, for at least about three weeks, for at least about one month, and/or throughout the patient's treatment). Acebilustat can, for example, be administered at a dose of about 50 mg every 12 or 24 hours (or once or twice a day), or at a dose of about 100 mg every 12 or 24 hours (or once or twice a day). In certain aspects, acebilustat is administered at a dose of 100 mg every 24 hours (or once a day). The total daily dose of acebilustat can be a dose that is about 200 mg or less, about 100 mg or less, about 50 mg or less. The total daily dose of acebilustat can also be from 100 mg to 200 mg, for example about 150 mg. The total daily dose of acebilustat can also be from about 50 mg to about 100 mg, for example, about 75 mg. In certain aspects, the dose of acebilustat is about 25 mg administered twice a day or a dose between about 25 and 50 mg administered twice a day. Acebilustat can be administered with or without food.

As discussed above, the acebilustat can be administered to a COVID-19 patient with mild to moderate disease. Mild to moderate disease is characterized by mild symptoms up to mild pneumonia. In yet additional aspects, the acebilustat is administered to a patient with mild disease. Mild disease can be characterized by the absence of viral pneumonia and/or hypoxia. In yet additional aspects, the acebilustat is administered to a patient that has not yet been hospitalized (e.g., before the severity of the disease warrants hospitalization).

As discussed above, the methods described herein reduce the risk of progression from mild to moderate disease to severe or critical disease. Treatment of severe disease and critical disease can entail oxygen therapy and/or use of ventilator. In yet additional aspects, the method is used to treat a patient who has moderate to severe COVID-19. As used herein, “severe COVID-19” encompasses both severe COVID-19 as well as critical COVID-19. Severe disease is characterized by dyspnea, hypoxia, or more than 50% lung involvement on imaging. Critical disease is characterized respiratory failure, shock, or multiorgan system dysfunction (cdc.gov/coronavirus/2019-ncov/hcp/clinical-guidance-management-patients.html). Symptoms of severe or critical disease include, for example, acute lung injury, ARDS, and multiple organ failure (Cavalcante-Silva et al. (2021). Neutrophils and COVID-19: The road so far. Int. Immunopharmacol. doi: 10.1016/j.intimp.2020.107233). Based on the large cohort study from China, the median time from symptom onset to dyspnea was 5 to 8 days in severe disease patients (cdc.gov/coronavirus/2019-ncov/hcp/clinical-guidance-management-patients.html). The median time from illness onset to ARDS was 8-12 days. In certain aspects, the acebilustat is administered to the patient within one or two days of the onset of symptoms and the treatment is continued for at least about a week, at least two weeks, at least 3 weeks, or at least about four weeks. The invention includes a method of decreasing or preventing aberrant immune response, or reducing the risk of aberrant immune response. Such aberrant immune response in COVID-19 has been described, for example, in Lucas et al. (2020), Longitudinal analyses reveal immunological misfiring in severe COVID-19, Nature 584: 463-469 and Hussman (2020), Cellular and Molecular Pathways of COVID-19 and Potential Points of Therapeutic Intervention, Frontiers in Pharmacology, https://doi.org/10.3389/fphar.2020.01169; the contents of which are expressly incorporated by reference herein). The aberrant immune response is characterized by elevated immune response, including an elevation of cytokine levels, throughout the course of the disease. Aberrant immune response can also be characterized by radiologic changes in lung tissues or cardiac tissue. Lucas et al. suggested that severe COVID-19 and late stage disease may be largely driven by the immune response to the virus and that it would be useful to inhibit cytokines represented in the immune response. The invention additionally includes a method of decreasing or preventing aberrant immune response, or reducing the risk of COVID-19 mediated lung injury, including COVID-19 mediated or COVID-19 induced ARDS or ALI. About 5% of COVID-19 patients progress to ARDS and/or multiorgan failure (Bime et al. (2020), Translational Research https://doi.org/10.1016/j.trs1.2020.12.008).

In certain aspects, the COVID-19 patient to be treated can be a patient, such as a mild or moderate patient, at risk for severe disease. Patients at risk for severe disease are patients who have one or more risk factors. Such risk factors include age. Mortality rates are highest in patients 80 or 85 years and older. In certain aspects, the patient is over 55, over 60, age over 65, age over 75, or over 85 years of age. The patient can additionally have one or more comorbidities that have been recognized as being a risk factor. Such comorbidities include cardiovascular disease, heart failure, coronary artery disease, cardiomyopathies, pulmonary hypertension, hypertension, Type II diabetes mellitus, chronic respiratory disease, COPD, Down Syndrome, cancer, prior stroke, obesity (BMI of 30 kg/m² or higher but less than 40 kg/m′) and severe obesity (greater than or equal to 40 kg/m²), sickle cell disease, and chronic kidney disease (cdc.gov/coronavirus/2019-ncov/hcp/clinical-guidance-management-patients.html and cdc.gov/coronavirus/2019-ncov/need-extra-precautions/people-with-medical-conditions.html?CDC_AA_refVal=https%3A%2F%2Fwww.cdc.gov%2Fcoronavirus%2F2019-ncov%2Fneed-extra-precautions%2Fgroups-at-higher-risk.html). Individuals that smoke are also at risk for severe disease. There is additionally some evidence that the following conditions also increase the risk of severe disease: moderate to severe asthma, cerebrovascular disease (affecting the blood vessels or blood supply to the brain), cystic fibrosis, immunocompromised state from blood or bone marrow transplant, immune deficiency, HIV, use of corticosteroid, neurologic conditions including, but not limited to dementia, liver disease, being overweight (BMI greater than 25 kg/m2 but less than 30 kg/m2), pulmonary fibrosis, thalassemia, and Type I diabetes mellitus dc.gov/coronavirus/2019-ncov/need-extra-precautions/people-with-medical-conditions.html?CDC_AA_refVal=https%3A%2F%2Fwww.cdc.gov%2Fcoronavirus%2F2019-ncov%2Fneed-extra-precautions%2Fgroups-at-higher-risk.html). In certain aspects, the patient is over 55 years of age and/or has at least one comorbidity. In other aspects, the patient has diabetes with a hemoglobin A1C>7.6%. In yet additional aspects, the patient has pre-existing pulmonary disease including, but not limited to, chronic obstructive pulmonary disease (COPD) and asthma. In additional embodiments, the patient has chronic kidney disease and/or a history of hypertension or cardiovascular disease. In yet further aspects, the patient has at least two comorbidities.

As discussed above, in some embodiments, the COVID-19 patient can be a patient suffering from moderate to severe COVID-19. Additionally, the methods can be used to treat a patient suffering late sequelae of COVID-19. The methods described herein can advantageously be used to reduce or replace corticosteroid treatment or administration in the moderate to severe patient or patient suffering from late sequelae. Corticosteroid therapy has been associated with decrease in viral clearance and can increase the risk of bacterial infections (Shah 2020, Can roflumilast become steroid-sparing alternative in the treatment of COVID-19? Med Hypotheses 144: 110246; RECOVERY Collaborative Group (2021), Dexamethasone in Hospitalized Patients with Covid-19. N Engl J Med. 2021 Feb 25;384(8):693-704. doi: 10.1056/NEJMoa2021436; covid19treatmentguidelines.nih.gov/immunomodulators/corticosteroids/). The methods described herein can reduce inflammation and/or reduce aberrant immune response and/or reduce lung injury (including, ARDS or ALI) in the patient that has moderate to severe COVID-19 or late sequelae with or without co-administration of a corticosteroid. Late sequelae of COVID-19 include persistent symptoms reported by certain patients that have been infected with COVID-19. These patients include those who initially experienced mild COVID-19 (cdc.gov/coronavirus/2019-ncov/hcp/clinical-care/late-sequelae.html). The most common reported symptoms of late sequelae include fatigue, dyspnea, cough, arthralgia, chest pain, cognitive impairment, depression, and headache (cdc.gov/coronavirus/2019-ncov/hcp/clinical-care/late-sequelae.html). Pulmonary function abnormalities have also been reported (cdc.gov/coronavirus/2019-ncov/hcp/clinical-care/late-sequelae.html; Wang et al. (2020), Long-Term Respiratory and Neurological Sequelae of COVID-19, Med Sci Monit. 2020; 26: e928996-1-e928996-10).

Acebilustat can be administered to a patient on top of their current treatment regime, or on top of the standard of care. It is understood that the early part of the disease is driven by replication of the SARS-CoV-2 virus and that the later part of the disease is driven by an exaggerated immune/inflammatory response to the virus (https://www.covid19treatmentguidelines.nih.gov/therapeutic-management/). The standard of care for the treatment of COVID-19 includes, but is not limited to, antiviral agents (including, but not limited to, Remdesivir) and anti-SARS-CoV-2 antibodies, including anti-SARS-CoV-2 monoclonal antibodies (for example, bamlanivimab, casirivimab, imdevimab, and any combination thereof). Treatment of COVID-19 can also include the use of convalescent plasma. Additionally, as discussed above, the treatment of severe COVID-19 or late sequelae of COVID-19 can also entail administration of a corticosteroid including, but not limited to, dexamethasone, prednisone, methylprednisolone, and hydrocortisone.

As used therein, a “therapeutically effective amount” or an “effective amount” refers to that amount of a compound or drug that, when administered to a mammal, preferably a human, is sufficient to effect treatment, as defined below, of a disease or condition of interest in the mammal, preferably a human. The amount of a compound of the invention which constitutes a “therapeutically effective amount” or an “effective amount” will vary depending on, for example, the activity of the specific compound employed; the metabolic stability and length of action of the compound; the age, body weight, general health, sex, and diet of the patient; the mode and time of administration; the rate of excretion; the drug combination; the severity of the particular disorder or condition; and the subject undergoing therapy, but it can be determined routinely by one of ordinary skill in the art having regard to his own knowledge and to this disclosure. In certain aspects, an effective amount or a therapeutically effective amount of acebilustat is an amount that inhibits LTA4h or inhibits LTB4, and/or that treats or inhibits or decreases the severity of the disease.

“Treating” or “treatment” as used herein covers the treatment of COVID-19 disease or associated disease or other condition of interest in a mammal, preferably a human, having the disease or condition of interest, and includes, for example: (i) inhibiting or decreasing the severity of the disease or condition, or one or more symptoms thereof, i.e., arresting or slowing development or progression of the disease or condition (including, for example, decreasing the risk of progression to severe disease; decreasing or reducing the risk of aberrant immune response; and/or decreasing or reducing the risk of COVID-19 mediated lung injury), and/or ameliorating one or more symptoms; (ii) relieving the disease or condition, i.e., causing regression of the disease or condition, or one more symptoms thereof; and/ or (iii) stabilizing the disease or condition. “Treating” or “treatment” in the context of COVID-19 can include decreasing the risk of progression; reducing, preventing, or decreasing the risk of COVID-19 mediated lung injury, and/or reducing, preventing, or decreasing the risk of aberrant immune response.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

A “pharmaceutical composition” refers to a formulation of a compound described herein, for example, acebilustat and/or an additional therapeutic agent, and a medium generally accepted in the art for the delivery of the biologically active compound to mammals, for example, humans. Such a medium includes all pharmaceutically acceptable carriers, diluents or excipients. “Optional” or “optionally” means that the subsequently described event of circumstances may or may not occur, and that the description includes instances where said event or circumstance occurs and instances in which it does not.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which, for example, has been approved by the United States Food and Drug Administration as being acceptable for use in humans or domestic animals.

Administration of the compounds or drugs described herein encompasses administration of a pharmaceutically acceptable salt of said compound or drug, for example, administration of a pharmaceutically acceptable salt of acebilustat. Administration of the compounds or drugs as described herein (such as acebilustat or other additional therapeutic agent), or their pharmaceutically acceptable salts, in pure form or in an appropriate pharmaceutical composition, can be carried out via any of the accepted modes of administration of agents for serving similar utilities. As described herein, the preferred mode of administration for acebilustat is oral administration. The pharmaceutical compositions described herein can be prepared by combining a compound or drug with an appropriate pharmaceutically acceptable carrier, diluent or excipient, and may be formulated into preparations in solid, semi-solid, liquid or gaseous forms, such as tablets, capsules, powders, granules, ointments, solutions, suppositories, injections, inhalants, gels, microspheres, and aerosols. Typical routes of administering such pharmaceutical compositions include, without limitation, oral, topical, transdermal, inhalation, parenteral, sublingual, rectal, vaginal, and intranasal. The term parenteral as used herein includes subcutaneous injections, intravenous, intramuscular, intrasternal injection or infusion techniques. Pharmaceutical compositions of the invention are formulated so as to allow the active ingredients contained therein to be bioavailable upon administration of the composition to a patient. Compositions that will be administered to a subject or patient take the form of one or more dosage units, where for example, a tablet may be a single dosage unit, and a container of a compound of the invention in aerosol form may hold a plurality of dosage units. Actual methods of preparing such dosage forms are known, or will be apparent, to those skilled in this art; for example, see The Science and Practice of Pharmacy, 20^{th} Edition (Philadelphia College of Pharmacy and Science, 2000). The composition to be administered will, in any event, contain a therapeutically effective amount of the compound or drug, or a pharmaceutically acceptable salt thereof, for treatment of a disease or condition of interest in accordance with the teachings of this invention.

A pharmaceutical composition can be in the form of a solid or liquid. In one aspect, the carrier(s) are particulate, so that the compositions are, for example, in tablet or powder form. In one aspect, the composition can be an encapsulated powder or granular form. In another aspect, an encapsulated powder or granular formulation can be opened and sprinkled in food or administered by gastric intubation. The carrier(s) can be liquid, with the compositions being, for example, an oral syrup, injectable liquid or an aerosol, which is useful in, for example, inhalatory administration. When intended for oral administration, the pharmaceutical composition can be in either solid or liquid form, where semi-solid, semi-liquid, suspension and gel forms are included within the forms considered herein as either solid or liquid.

As a solid composition for oral administration, the pharmaceutical composition may be formulated into a powder, granule, compressed tablet, pill, capsule, chewing gum, wafer or the like form. Such a solid composition will typically contain one or more inert diluents or edible carriers. In addition, one or more of the following may be present: binders such as carboxymethylcellulose, ethyl cellulose, microcrystalline cellulose, gum tragacanth or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin; a flavoring agent such as peppermint, methyl salicylate or orange flavoring; and a coloring agent.

When the pharmaceutical composition is in the form of a capsule, for example, a gelatin capsule, it may contain, in addition to materials of the above type, a liquid carrier such as polyethylene glycol or oil.

The pharmaceutical composition can be in the form of a liquid, for example, an elixir, syrup, solution, emulsion or suspension. The liquid can be for oral administration or for delivery by injection, as two examples. When intended for oral administration, a composition can contain, in addition to the present compounds, one or more of a sweetening agent, preservatives, dye/colorant and flavor enhancer. In a composition intended to be administered by injection, one or more of a surfactant, preservative, wetting agent, dispersing agent, suspending agent, buffer, stabilizer and isotonic agent may be included.

The liquid pharmaceutical compositions of the invention, whether solutions, suspensions or other like form, can include one or more of the following adjuvants: sterile diluents such as water for injection, saline solution, preferably physiological saline, Ringer's solution, isotonic sodium chloride or physiological saline, fixed oils such as synthetic mono or diglycerides which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. Physiological saline is a preferred adjuvant. An injectable pharmaceutical composition is preferably sterile.

While this invention has been particularly shown and described with references to preferred embodiments thereof, it will be understood by those skilled in the art that various changes in form and details may be made therein without departing from the scope of the invention encompassed by the appended claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference. The relevant teachings of all patents, published applications and references cited herein are incorporated by reference in their entirety.

What is claimed is:

1. A method for treating a patient that is or has been infected with COVID-19, the method comprising administering to said patient an effective amount of acebilustat at least once a day.

2. The method of claim 1, wherein the method comprises reducing or preventing an aberrant immune response, or decreasing the risk of aberrant immune response, in the patient.

3. The method of claim 2, wherein the aberrant immune response comprises radiologic changes in lung tissues.

4. The method of claim 2, wherein the aberrant immune response comprises radiologic changes in cardiac tissues.

5. The method of claim 2, wherein the aberrant immune response comprises acute respiratory distress syndrome (ARDS).

6. The method of claim 2, wherein the aberrant immune response comprises acute lung injury (ALI).

7. The method of claim 2, wherein the aberrant immune response comprises multisystem organ failure (MSOF).

8. The method of claim 2, wherein the patient has mild or moderate disease.

9. The method of claim 8, wherein the patient has mild disease.

10. The method of claim 1, wherein the patient has one or more risk factors for severe disease.

11. The method of claim 1, wherein the patient is over 55 years old.

12. The method of claim 11, wherein the patient is over 65 years old.

13. The method of claim 10, wherein the patient has at least one comorbidity.

14. The method of claim 13, wherein the at least one comorbidity is selected from the group consisting of cardiovascular disease, heart failure, coronary artery disease, cardiomyopathies, pulmonary hypertension, hypertension, Type II diabetes mellitus, chronic respiratory disease, COPD, Down Syndrome, cancer, prior stroke, obesity and severe obesity, sickle cell disease, and chronic kidney disease.

15. The method of claim 13, wherein the at least one comorbidity is selected from the group consisting of asthma, cerebrovascular disease (affecting the blood vessels or blood supply to the brain), cystic fibrosis, immunocompromised state from blood or bone marrow transplant, immune deficiency, HIV, use of corticosteroid, neurologic conditions including, but not limited to dementia, liver disease, being overweight, pulmonary fibrosis, thalassemia, and Type I diabetes mellitus.

16. The method of claim 10, wherein the risk factor is pre-existing pulmonary disease.

17. The method of claim 16, wherein the pulmonary disease is COPD or asthma.

18. The method of claim 10, wherein the risk factor is selected from pre-existing chronic kidney disease, diabetes, hypertension, and/or cardiovascular disease.

19. The method of claim 8, wherein the patient has not yet been hospitalized for COVID-19.

20. The method of claim 1, wherein the acebilustat is administered orally.

21. The method of claim 20, wherein the acebilustat is administered at a total daily dose of about 200 mg or less.

22. The method of claim 21, wherein the acebilustat is administered at a total daily dose about 100 mg or less.

23. The method of claim 20, wherein the acebilustat is administered once or twice a day.

24. The method of claim 1, wherein the method comprises reducing or preventing COVID-19-mediated lung injury, or decreasing the risk of COVID-19-mediated lung injury.

25. The method of claim 24, wherein the subject is symptomatic.

26. The method of claim 1, wherein the method reduces the risk of COVID-19 progression.

27. The method of claim 1, wherein the patient is suffering from moderate to severe COVID-19.

28. The method of claim 1, wherein the patient is suffering from late sequelae of COVID-19.
